(19) United States
(12) Patent Application Publication
(10) Pub. No.: US 2013/0183391 A1
(43) Pub. Date: Jul. 18, 2013

(54) Palm fiber-based dietary supplements

(71) Applicants: Alexander G. Schauss, Tacoma, WA (US); Winson Fong, Telok Panglima Garang (MY)

(72) Inventors: Alexander G. Schauss, Tacoma, WA (US); Winson Fong, Telok Panglima Garang (MY)

(21) Appl. No.: 13/791,518

(22) Filed: Mar. 8, 2013

Related U.S. Application Data
(62) Division of application No. 11/190,296, filed on Jul. 27, 2005.
(60) Provisional application No. 60/591,277, filed on Jul. 27, 2004.

(51) Int. Cl. A61K 36/889

(52) U.S. Cl. CPC A61K 36/889
USPC 424/727

(57) ABSTRACT

The present invention relates to palm fiber-based dietary supplements. Specifically, the inventions relates to compositions of palm trunk fiber with high antioxidant capability and SOD-like activity, and their uses. The invention further provides for methods of preventing or treating disorders in a subject caused by reactive oxygen chemical species.

### RELATED US APPLICATION(S)

The present application claims priority to U.S. Application Ser. No. 60/591,277, filed Jul. 27, 2004, which application is hereby incorporated herein by reference.

### FIELD OF THE INVENTION

The present invention relates to methods of making palm fiber-based dietary supplements, and uses thereof.

### BACKGROUND OF THE INVENTION

Dietary fiber is the part of food that resists digestion and is found only in plant foods. Grain products, vegetables, legumes, fruits, nuts and seeds are all rich in dietary fiber. Dietary fiber includes several different types of compounds, e.g., gums, mucilages, pectins, lignin, cellulose and hemicelluloses. Generally, dietary fiber is not a source of calories or vitamins or minerals. There are two general categories of dietary fiber, e.g., insoluble dietary fiber and soluble dietary fiber. Soluble dietary fiber dissolves in water, e.g., gums and gels. Insoluble dietary fiber is a coarse, chewy material that will not dissolve in water, i.e., commonly known as roughage.

Insoluble dietary fiber and soluble dietary fiber are both important for health. Ingesting soluble fiber help control diabetes and reduce blood cholesterol. On the other hand, ingesting insoluble dietary fiber aids in bowel regularity, prevents intestinal disorders (e.g., spastic colon and diverticulitis), cancer (e.g., colon cancer). Some edible plant materials are better sources of one form of dietary fiber than the other for of dietary fiber. For example, soluble fiber accounts for half of the fiber in oat bran but only a fifth of the fiber in wheat bran.

There is a need for the identification of natural product with high dietary fiber useful in the production of dietary supplement compositions and useful in methods to prevent or treat disease.

### BRIEF SUMMARY OF THE INVENTION

The present invention relates to the identification of palm fiber-based dietary supplement with high Oxygen Radical Absorbance Capacity scores (ORAC) and super oxide dismutase-inhibitory activity.

These and other objects of the present invention will be apparent from the detailed description of the invention provided below.

### BRIEF DESCRIPTION OF THE DRAWINGS

The invention will be more fully understood by reference to the following drawings which are for illustrative purposes only:

FIG. 1 is a graph comparing the hydrated fecal weight of animals fed palm fiber with the hydrated fecal weight of animals fed other natural products.

FIG. 2 is a graph comparing the fecal bulking indices of various dietary fiber sources.

### DETAILED DESCRIPTION OF THE INVENTION

It is to be appreciated therefore that certain aspects, modes, embodiments, variations and features of the invention described below in various levels of detail in order to provide a substantial understanding of the present invention. In general, such disclosure provides beneficial dietary supplement compositions, combinations of such compositions with other dietary supplement compositions, and related methods of producing and using same. The references cited throughout this application are incorporated herein by reference in their entireties.

Accordingly, the various aspects of the present invention relate to therapeutic or prophylactic uses of certain particular dietary supplement compositions in order to prevent or treat a disease or an injury induced by pathological free radical reactions. The various aspects of the present invention further relate to therapeutic or prophylactic uses of certain particular dietary supplement compositions in order to prevent or treat a disease or an injury associated with decreased SOD activity. Accordingly, various particular embodiments that illustrate these aspects follow.

It is to be appreciated that the various modes of treatment or prevention of medical conditions as described are intended to mean “substantial”, which includes total but also less than total treatment or prevention, and wherein some biologically or medically relevant result is achieved.

A “subject” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of a composition, as used herein, is a quantity sufficient to achieve a desired therapeutic and/or prophylactic effect, for example, an amount which results in the prevention of or a decrease in the symptoms associated with a disease that is being treated. The amount of composition administered to the subject will depend on the type and seventy of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compositions of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compositions of the present invention can also be administered in combination with each other, or with one or more additional compositions, i.e., actives or excipients.

An “African Oil Palm (Palmaceae Elaeis guineensis Jacq.),” as used herein, is a spineless palm tree native to western Africa. This is a tropical tree of great economic importance throughout the world. Fruits and seeds of E. guineensis yield a valuable vegetable oil, widely employed for nutritious, cosmetic and trade purposes. For this reason, this palm is widely cultivated not only throughout Africa, its area of origin, but also in other countries, such as Antilles, South America, Malaysia, Indochina, etc., where it is grown in large plantations. For example, in Malaysia, about 2.2 million hectares of land and under oil palm cultivation; at 130 palms per hectare, there are about 286 million palms.

The fruits of E. guineensis are borne in large bunches, each of which may carry up to 20 pounds of fruit. The red fruits are oval, 1 to 2 inches long and an inch or more in diameter. The flesh contains 30 to 70 percent of nondrying oil. The seed also contains oil, the palm kernel oil of commerce. Oil palm fiber is rich in cellulose, hemi-cellulose and lignin and useful for the inclusion in the diet of mammals. The fiber can be dried and pelleted to improve its shelf-life and decrease it bulkiness. Such techniques are widely known.

### Antioxidant Potential of the Palm Fiber Composition of the Invention

Over the past few decades, free radicals have come to be appreciated increasingly for their importance to human health and disease. Many common and life-threatening diseases, including atherosclerosis, cancer, and aging, have free radical reactions as an underlying mechanism of injury. Over this period of time, our conceptual understanding of the interaction of free radicals with living organisms has evolved and provided unprecedented opportunities for improving the quality and even length of human life.

One of the most common types of free radicals are the reactive oxygen species (ROS). These are the products of normal cell respiration and metabolism and are generally regulated by antioxidants produced in the body. Due to environmental agents such as pollution, and lifestyle factors such as smoking or exercising, the production of free radicals is increased. Such increase may bring the body out of balance, especially as the body ages and the mechanisms that produce antioxidants lose their ability to produce these compounds at their necessary rate, resulting in oxidative stress. The resulting damage can range from disruption of biological processes, killing of cells, and mutation of genetic material, which may lead to the occurrence of cancer.

The potential use of dietary supplements for protection against the effects of oxidative stress and the progression of degenerative diseases and aging has been the subject of an increasing number of studies during the past two decades. In the market today there are many products that contain antioxidant at various levels. These come in the form of foods, liquids and nutritional supplements. The richest sources of these vital nutrients commonly are found in fruits and vegetables having compounds such as Vitamin C, Vitamin E, beta-carotene and others.

The antioxidant hypothesis postulates that supplementation with dietary antioxidants can alleviate the redox imbalance associated with disease. Antioxidants function to bind these free radicals and stabilize and scavenge them out of the system, thereby reducing the amount of damage free radicals may cause.

Synthetic antioxidants such as BHA (butylated hydroxy anisole), BHT (butylated hydroxy toluene) and NDGA (nordihydro-guaiaretic acid) have been developed to date. By way of examples of natural antioxidants, there are antioxidant enzymes such as superoxide dismutase (SOD), peroxidase, catalase and glutathione peroxidase, and non-enzymatic antioxidant substances such as tocopherol (vitamin E), ascorbic acid (vitamin C), carotenoid and glutathione.

However, synthetic antioxidants may cause allergic reactions and oncogenesis due to their strong toxicity in the body, and be easily disrupted by heat due to temperature sensitivity. On the other hand, natural antioxidants are safer than synthetic antioxidants in the body but have the problem of weak effect. Therefore, the development of a new natural antioxidant having no problem with safety in use and also having excellent antioxidant activity has been required.

In the market today there are many products that contain antioxidants at various levels. These come in the form of foods, liquids and nutritional supplements. The richest sources of these vital nutrients commonly are found in fruits and vegetables. Antioxidants function to bind these free radicals and stabilize and scavenge them out of the system, thereby reducing the amount of damage free radicals may cause.

It is important to be able to assess the ability of antioxidants in these foods to absorb free radicals. USDA Researchers at Tufts University developed a laboratory test know as ORAC (Oxygen Radical Absorbance Capacity) which rates different foods according to their antioxidant content and its ability to bind these free radicals. Through this test, different foods may be compared and analyzed for their antioxidant ability.

As detailed below, the present invention identifies compositions of trunk tissue of palm trees, e.g., the African Oil Palm (E. guineensis), as having antioxidant activity as judged by significant ORAC scores as well as SOD-like activity. Palm fiber was not previously known to have antioxidant activity. Specifically, the present invention provides a palm fiber-based dietary supplement composition with significant ORAC scores and SOD-like activity. As a result of the present invention, it is now apparent that the palm fiber provides a very good source for a dietary supplement with high antioxidant activities against hydroxy radical, peroxynitrite and super oxide.

### I. Analysis of a Palm Fiber Composition Against Hydroxyl Radical and Peroxynitrite

### A. General

### 1. Hydroxyl Radical

Hydroxyl radical is highly reactive and is known to destroy molecules and tissues. It reacts at diffusion rates with virtually any molecule found in its path including macromolecules such as DNA, membrane lipids, proteins, and carbohydrates. In terms of DNA, the hydroxyl radical can induce strand breaks as well as chemical changes in the deoxyribose and in the purine and pyrimidine bases.

Damaged proteins, many of them crucial enzymes in neurons, lose their efficiency and cellular function wanes. Protein oxidation in many tissues, including the brain, has been proposed as an explanation for the functional deficits associated with aging. The palm fiber compositions described herein protect a mammal against oxidative damage due to hydroxyl radicals, when the palm fiber compositions are administered to the mammal as described.

### 2. Peroxynitrite

Peroxynitrite is a cytotoxic product of nitric oxide (NO) and superoxide. Peroxynitrite is a far stronger oxidant and much more toxic than either nitric oxide or superoxide acting separately. The palm fiber compositions described herein protect a mammal against damage due to nitric oxide, superoxide, and peroxynitrite, when the palm fiber compositions are administered to the mammal as described.

A variety of pathologies are associated with the formation of peroxynitrite, a potent oxidant formed from the reaction of NO with superoxide. This reaction is the fastest reaction NO is known to undergo, and transforms two relatively unreactive radicals into a more reactive oxidant, peroxynitrite. Peroxynitrite is invariably formed in larger amounts when more NO is produced, and/or when an elevated level of O₂⁻ prevails.

Peroxynitrite is a potent oxidant implicated in a number of pathophysiological processes. Peroxynitrite freely travels across cellular lipid membranes. The calculated permeability coefficient for peroxynitrite compares well with water and is approximately 400 times greater than superoxide, hence is a significant biological effector molecule not only because of its reactivity but also its diffusibility. (Lee, J., Marla, S. S. Peroxynitrite rapidly permeates phospholipid membranes. Proc Natl Acad. Sci., 1997.)

In this regard, pathologies such as diabetes, atherosclerosis, and ischemia-reperfusion injury, are associated with oxidative stress characterized by an elevated level of O₂⁻ that can lead to increased peroxynitrite formation. Recent evidence also suggests multiple sclerosis and Alzheimer's disease are associated with peroxynitrite formation. In addition, peroxynitrite has also been implicated during ischemia and reperfusion, and during sepsis and adult respiratory distress syndrome. Ischemia and reperfusion are accompanied by an increase in superoxide due to the activation of xanthine oxidase and NAPDH oxidase, respectively. Thus, peroxynitrite is likely to be implicated in a number of pathologies in which an imbalance of NO and O₂⁻ occurs. The formation of peroxynitrite is desirable for non-specific immunity but possibly not during signaling by NO.

Peroxynitrite is formed in biology from the reaction of nitric oxide and superoxide. The enzyme SOD lowers superoxide and prevents peroxynitrite formation (see my review: Pryor, W. A. and Squadrito, G. L. (1995). Am. J. Physiol. (Lung Cell. Mol. Physiol. 12) 268, L699-L722). The chemistry of peroxynitrite: a product from the reaction of nitric oxide with superoxide). Peroxynitrite is a potent oxidant and itself can oxidize many biomolecules. Nevertheless, in biological systems, it reacts mostly with carbon dioxide to form reactive intermediates, such as ONOOCO₂⁻, O₂NOCO₂⁻, COO₃⁻, and NO₂. Of these intermediates, only COO₃⁻ and NO₂ participate in bimolecular reactions with biological target molecules; the CO₂ adducts ONOOCO₂⁻ and O₂NOCO₂⁻ are too short lived and decompose before they can react bimolecularly.

Oxidative stress, such as that caused by peroxynitrite is known to damage the vascular endothelium, a process that can lead to atherosclerosis (Thom, S. R. and Ischiropoulos, H. Mechanism of oxidative stress from low levels of carbon monoxide. Health Effects Institute Research Report, number 80, 1997.)

### B. ORAC Assay

The ORAC Assay was developed by Cao et al., and first reported in 1993 (Cao G, et al., Free Rad. Biol. Med. 1993:14:303-11). The ORAC assay measures the free-radical quenching capability of test compositions. The HORAC assay measures the quenching capability against hydroxyl radical. The NORAC assay measures the quenching capability against peroxynitrite. As detailed above, these radicals can be extremely harmful in vivo.

Each of these assays is a quantitative measure of the ability of foods, blood and antioxidant blends to subdue oxygen free radicals in vitro. The HORAC primarily reflects metal-chelating radical prevention ability against hydroxyl radical formation, and the NORAC reflects peroxyl radical absorption capacity. It is therefore, expected that the samples with high HORAC values do not necessarily have high ORAC values and vice versa. Of all foods tested to date, HORAC values range from 15 (apple powder) to 333 (elderberry). Boxin, O. et al., J. Agric. Food Chem. 2002, 50: 2772-2777.

Test samples of palm-fiber composition of the invention, derived from the African Oil Palm (E. guineensis), were analyzed for antioxidant activity at Brunswick Laboratories (Wareham, Mass.) using an automated ORAC method (Cao et al., CLINICAL CHEMISTRY, 41(12), 1738-44 (1995); Ou et al., J. Agric. Food Chem., 50, 2772-77 (2002). Brunswick Laboratories, working with the USDA, introduced a new fluorescence probe, fluorescein, which has been tested with several hundred samples, in side-by-side comparison with beta-Phycoerythrin. Fluorescein, unlike beta-PE, does not interact with the tested samples, and being a synthetic compound, fluorescein has no measurable variability from lot-to-lot. Most importantly, samples tested multiple times under the same conditions maintain consistent and repeatable results. The development of the ORAC assay using fluorescein as the fluorescence probe has been conducted in cooperation with the developers of the original automated ORAC Assay, where beta-PE was utilized as the fluorescence probe. Based on the extensively mechanistic studies, the fluorescein based ORAC assay as being the new standard ORAC procedure. The two ORAC assays are distinguished herein by using the subscripts PE for phycoerythrin, and FL for fluorescein-ORAC_{PE} and ORAC_{FL}.

### C. Results

The antioxidant activity of palm fiber powder (Brunswick Lab ID. Brunswick Laboratories, Wareham, Mass.) was determined by ORAC analysis technique (as detailed above) and is summarized below in Table 1.

TABLE 1
Measurement of Antioxidant Activities Against
Hydroxyl Radical and Peroxynitrite

| Samples | HORAC | NORAC |
|---|---|---|
| Palm fiber | 32.55 | 2.86 |

The HORAC result in Table 1 is expressed as milligrams caffeic acid equivalents per gram. The NORAC result in Table 24 is expressed as micromole Trolox equivalent per gram.

Table 2 summarizes the antioxidant activity of palm fiber as determined by ORAC-hydro_{FL} analysis technique (Brunswick Laboratories, Wareham, Mass.; as detailed above).

TABLE 2
Measurement of Antioxidant Activities Against Hydroxyl Radical

| | Brunswick | ORAC_{hydroFL}* | ORAC_{hydroFL}* | ORAC_{totalFL} |
|---|---|---|---|---|
| Sample ID | Lab ID | (μmoleTE/g) | (μmoleTE/g) | (μmoleTE/G) |
| Palm | 03-2065 | 90 | 3 | 93 |
| fiber-based | | | | |
| dietary | | | | |
| composition | | | | |
| (MAL001) | | | | |

*The ORAC analysis, which utilizes Fluorescein as the fluorescent probe, provides a measure of the scavenging capacity of antioxidants against the peroxyl radical, which is one of the most common reactive oxygen species (ROS) found in the body. ORAC_{hydro} reflects water-soluble antioxidant capacity and the ORAC_{hydro} is the lipid soluble antioxidant capacity. ORACₜₒₜₐₗ is the sum of ORAC_{hydro} and ORACₗᵢₚₒ. Trolox, a water-soluble Vitamin E analog Vitamin Eanalog, is used as the calibration standard and the ORAC result is expressed as micromole Trolox equivalent(TE) per gram.

### II. Analysis of Sod-Like Activity in the Palm Fiber Composition of the Invention

### A. General

It is estimated one percent of total oxygen consumed by an adult (70 kg body mass) is converted to superoxide anion. An adult at rest utilizes 3.5 mL O₂/kg/min, which would result in 0.147 mole/day O₂⁻. O₂⁻ is believed to be cause of other reactive oxygen species such as hydrogen peroxide, peroxynitrite, and hydroxyl radicals (from hydrogen peroxide). Therefore, O₂⁻ scavenging capacity in human body is the first defense line against oxidative stress. In fact, it is reported that over-expression of SOD and catalase in transgenic flies extended life-span by as much as one-third, perhaps, due to decreased oxidative stress reflected by lower protein carbonyl contents (Orr and Sohal, Science 263: 1128-1130, 1994. The palm fiber compositions described herein protect a mammal against damage due to superoxide anions, when the palm fiber compositions are administered to the mammal as described.

Superoxide scavenging capacity in blood is a very important parameter for one's antioxidant status. This assay is designed far accurately quantify this parameter in a high throughput fashion.

### B. Superoxide (O₂⁻) Scavenging Activity Assay (SOD) Procedure

### 1. Instruments

Precision 2000 eight channel liquid handing system and Synergy HT microplate UV-VWAS and fluorescence reader both from Bio-tek Inc. (Winooski, Vt.).

### 2. Reagents

Hydroethidine was from Polysciences, Inc. (Warrington, Pa.). Xanthine oxidase (from butter milk, Catalog number X4875), xanthine, SOD (from bovine erythrocytes, catalog number S 2515) were purchased from Sigma-Aldrich (St. Louis, Mo.).

Buffer. The buffer consists of 75 mM phosphate buffer (pH 7.4) containing 100 μM diethylenetriamine pentaacetic acid (DTPA). To prepare the buffer, 0.0393 g of diethylenetriamine (DTPA) was weighed out and 10 ml of ORAC buffer working solution was added. This yielded 10 mL of 10 mM DTPA stock solution. Next, to 198 ml of ORAC buffer working solution was added 2 mL of DTPA stock solution. This yielded 200 mL of 100 μM O₂⁻ buffer working solution with DTPA.

Xanthine oxidase. The xanthine oxidase suspension (in refrigerator) from Sigma was diluted 20 times by buffer to give a homogeneous solution.

Take 19 ml of O₂⁻ buffer and add 1.0 mL of Xanthine oxidase suspension. This yielded 20 ml of Xanthine oxidase working solution, which was made fresh daily.

Xanthine solution. Xanthine (15 mg) was weighed and place in a clear glass bottle. 5 ml of 0.1 N sodium hydroxide (0.1N NaOH) was added and the solution was vortexed and sonicated until the solid was dissolved. 95 mL of O₂ buffer was added and vortexed. This yielded 100 ml of Xanthine solution. The solution was kept at room temperature to avoid precipitation of xanthine. The Xanthine solution was made fresh daily.

Hydroethidine (HE) Working Solution. Stock solution of dihydroethidium—0.04 g of dihydroethidium was added to 20 mL of acetonitrile. This yielded 20 ml of HE stock solution (2 mg/mL), which was stored in small aliquot vials at −80° C. Next, 0.125 mL of dihydroethidium (HE) stock solution was added to 24.875 ml of xanthine solution. The solution was sonicated and heated until clear. This yielded 25 ml of Hydroethidine (HE) working solution, which was prepared fresh daily.

SOD Working Solution. Thirty thousand units of SOD (Sigma) was reconstituted in ten mL buffer solution. The solution was divided into small aliquots (0.4 mL per vial, stock solution) and kept at −20° C. This yielded 3000 units, which was diluted to 30 units for use (see below). 200 μL of SOD 3000 unit stock solution was added to 19.8 ml of O₂⁻ buffer to yield 20 ml of SOD 30 unit working solution.

Control. The stock solution was Manganese (III) 5,10,15,20 tetrachloride stock solution 1144 μM which was stored at −80° C. To prepare the working solution, the stock solution was diluted 100-fold with O₂⁻ buffer and vortexed. By taking 9.9 ml of O₂⁻ buffer and adding 100 μL of Manganese stock solution, 10 mL of 11.44 μM Manganese working solution, which was placed in wells G1 and G12 as controls.

### 3. Assay Procedures

The assay was carried out on a Precision 2000 liquid handling system with a 96-well microplate using the following protocol:
In plate one (polypropylene) 200 μL of samples were added to wells B1, C1, E1, F1, and B12, C12, E12, F12.
200 μL of SOD working solution was added to D1 and D12 wells.
200 μL of O₂⁻ buffer was added to A1, H1, A12, and H12 wells.
200 μL of Manganese working solution was added to G1 and G12.
The reagents were loaded into the cups on rack B of the precision 2000 as follows:
   20 ml of O₂⁻ Buffer in B1
   20 ml of HE in B2
   20 ml of Xanthine oxidase in B4
A ×2 dilution (ORAC ×2) was carried out on a Precision 2000. A dilution was carried out so that all the samples, standard, and blank were diluted by 2, 4, 8, 16, and 32 times.
25 μl of the solutions in each well were transferred to a reaction plate (polystyrene, 320 μL) followed by the addition of 150 μL HE working solution.
Incubate reading plate for 20 min at 37° C.
After incubation, add Xanthine oxidase by running AAPH addition (B4) program. This allows 25 μL Xanthine oxidase working solution to be added to all wells in plate #2.
After xanthine oxidase was added, place plate in plate reader.
The plate and the fluorescence was read every minute for ten minutes with excitation filter at 485±25 nm and emission filter at 590±30 nm the readings were referenced to low well of D1 arbitrarily set at 5000 units. Plate two layout (polystyrene) each well contains 150 μl. HE working solution, 25 μL sample, and 25 μL xanthine oxidase (added after 30 min. preheat).

### 4. Data Processing

From the raw data, a linear curve was obtained and the slopes of the curves were calculated by the KC-4 program used to control the plate reader. The slopes were exported and further calculations were executed by Microsoft Excel software.

### 5. Simplified Chemical Kinetics

O₂⁻ was generated constantly by the following reaction catalyzed by xanthine oxidase. The rate of superoxide production was constant and pseudo-zero order to xanthine, which was in large excess in comparison with xanthine oxidase.

xanthine+O₂→uric acid+O₂⁻ (1)

The superoxide formed was either reacted will HE or scavenged by SOD.

HE+O₂⁻→Oxidized HE (2)

2O₂⁻→O₂+H₂O₂ (3)

O₂⁻+Sample P (4)

Assuming steady state concentration of O₂⁻ the fluorescence increase rates in the absence (Vo) and presence (V) of O₂⁻ scavenger (SOD) have the following relationship:

Vₒ/V=1+k₃[SOD]/(k₂·[HE]) (5)

The plot of VoN vs [SOD] will give a linear curve with interception at (0, 1) and slope k₃/k₂[HE]. For an unknown sample the ratio between the slopes of the unknown and the standard was:

{k3/k2[HE]}/{kₛ/k₂[HE]}=k₃/kₛ (6)

Equation (5) would give relative SOD activity of a sample with unit of measure of SOD unit equivalent per gram or per liter of the sample depending on the concentrations used in plotting a sample's Vₒ/V vs concentration curve.

### C. Results

The superoxide anion scavenging potential of palm fiber-based dietary composition of the invention, derived from the African Oil Palm (E. guineensis), was measured as detailed above (Brunswick Lab ID. Brunswick Laboratories, Wareham, Mass.). The most studied SOD from a natural source is wheat sprout SOD. The SOD activity for wheat sprout is 160 to 500 unit per gram basis. By comparison, the palm fiber-based dietary composition of the invention was substantially high in superoxide scavenging capability (i.e., SOD-like activity) as summarized below in Table 3.

TABLE 3
SOD-Like Activity of the Palm fiber-based dietary composition

| Sample | SOD (unit/g)* |
|---|---|
| palm fiber-based dietary composition | 400 |

Result is expressed as SOD unit equivalent per gram

### Preparation of the Palm Fiber-Based Dietary Composition of the Invention

A palm fiber-based dietary composition was prepared from the African Oil Palm trees (E. guineensis) essentially as described in Japanese Patent Application No. 983491995, filed Dec. 5, 2000. Briefly, palm oil trees were pushed down with the end of a bulldozer put at positions of 3 to 4 m height from the ground of the trunks, and the trunks were cut by a chain saw at positions of 1 to 2 m upper from the roots, to be separated into roots and trunk. Palm oil trunk logs were carried to a sawmill, and sawn into a square timber and a plate, respectively, by a sawmill machinery. The sawn materials and logs were ground into large sawn dust, and fibrovascular bundle and dietary fiber were dried under natural environment or with hot air until a water content of 6 to 18%, then, ground by a fine grinding machine. Subsequently, they were sieved into 35 to 400 mesh, obtaining a dietary fiber palm oil trunk powder containing 70% or more of dietary fiber and including insoluble hemicellulose, pectin, cellulose, lignin, mucin and mucus. The degree of drying is appropriately set depending on the condition of water content of the palm oil trunk ground material, and it is suitable that dietary fiber is contained in large amount after drying and the water content of the palm oil trunk powder is 18% or less. This condition was necessary for distribution of the product and also for preventing corrosion of the palm oil trunk powder. Other sources of palm fiber useful in palm fiber-based dietary compositions and methods of the present invention include, e.g., but are not limited to, Acoelorrhaphe wrightii; Actinorhytis calapparia; Archontophoenix alexandrae; Areca catechu; Arenga hastata; Arenga hookeriana; Arenga undulatifolia; Bentinckia nicobarica; Bismarckia nobilis; Butia capitate; Calyptrocalix ghiessbretannia; Carpentaria acuminate; Caryota gigas; Caryota mitis; Caryota no; Chamadorea seizifrii; Chamaerops humilis; Chrysalidocarpus lucubensis; Chrysalidocarpus lutecens; Chrysalidocarpus madagascarensis; Coccothrinax crinita; Copemicia alba; Copemicia prunifera; Copemicia rigida; Corypha umbraculifera; Cyrtostachys renda; Daemonorops mollis; Dypsis decaryl; Dypsis decipiens; Elaeis guineensis; Hyophorbe lagenicaulis, Hyophorbe vershaffeltii; Hyphaene thebaica; Johannesteijsmannia altifrons; Kerriodoxa elegans; Latania lontaroides; Licuala elegans; Licuala grandis; Licuala spinosg Livistona Cape River, Livistona chinensis; Livistona decipiens; Livistona drudei; Livistona mariae; Livistona muelleri; Livistona rotundifolia; Livistona saribus; Metroxylon sagu; Neodysis lastelliana; Normanbya nomranbyi; Oncosperma tigillarium; Phoenix daclylifera; Phoenix humilis; Phoenix paludosa, Phoenix pusilla; Phoenix reclinata; Phoenix roebeienii, Phoenix rupicola; Phoenix sylvestris; Pritchardia pacifica; Pritchardia thurstonii, Ptychosperma macarthurii; Ptychosperma microcarpum; Raphia regalis; Ravanea rivularis; Rhapis excelsa; Rhapis laosensis; Roystonea regia Sabal palmetto; Trachycarpus fortunei; Veitchia merrillii; Wallichia disticha; Washingtonia robusta; Wodyetia bifurrata. Extraction of fiber from these plants will be known to those of skill in the art. Compositions of fiber from these plants can be assayed for antioxidant potential as described above. The compositions thus provide protection for a mammal against damage due to nitric oxide, superoxide, and peroxynitrite, when the compositions are administered to the mammal.

Tables 4-8 One example of components of palm dietary fiber powder of present invention as determined by separations and analyses

TABLE 4
Proximate composition and dietary fiber of palm fiber

| | Percent % | Percent % |
|---|---|---|
| Moisture | | 6.10 |
| Protein (Kjeldahl as N = 0.55%) | | 3.44 |
| Ash | | 3.00 |
| Oil extract total | | 7.45 |
| Fats in oil extract | 4.16 | |
| Phenolics in oil extract | 3.29 | |
| Hydrophilic extract total | | 8.74 |
| Sugar | 6.24 | |
| Phenolics | 2.50 | |
| Klasen lignin | | 7.30 |
| Total in proximate composition | | 36.03 |
| Dietary fiber content of palm fiber | | |
| Insoluble fiber | | 52.00 |
| Soluble fiber from ethanol precipitation | | 7.00 |
| Soluble fiber in ethanol supernatant | | 4.00 |
| Total dietary fiber | | 63.00 |
| Total accounted from fractionation | | 99.03 |

TABLE 5
Oils Composition of the palm fiber
Lipophillic extract - 4.16% fatty acids
Gas Chromatography
Fatty acid methyl esters

| Fatty Acids | Percent % |
|---|---|
| C-6 through C-10 | <0.50 |
| C-12:0 | 0.20 |
| C-14:0 | 1.10 |
| C-16:0 | 40.10 |
| C-16:1 | 0.30 |
| C-18:0 | 4.30 |
| C-18:1 | 42.30 |
| C-18:2 | 8.40 |
| C-18:3 | 0.50 |
| C-20:0 | 0.40 |
| Total | 100.00 |

TABLE 6
Hydrophilic extractables of defatted palm fiber
Sugars in the 80% ethanol extracts by high
performance liquid chromatography - 6.24%

| Sugar | Percent % |
|---|---|
| Sucrose | 25.00 |
| Glucose | 35.00 |
| Fructose | 40.00 |
| Total | 100.00 |

TABLE 7
Neutral sugar carbohydrate composition
Gas chromatography of alditol acetates

| | Insoluble | Soluble |
|---|---|---|
| Sugar | fiber % | fiber % |
| rhamnose | 2.50 | 1.50 |
| arabinose | 27.00 | 17.00 |
| xylose | 43.00 | 9.00 |
| mannose | 7.00 | 38.00 |
| galactose | 5.00 | 16.00 |
| glucose | 14.00 | 18.00 |

TABLE 8
Polysaccharide components of defatted,
desugared palm fiber 63% of total

| | Percent % | Percent % |
|---|---|---|
| Pectin total | | 18.00 |
| Isolated soluble | 3.00 | |
| fiber | | |
| Isolated insoluble | 7.00 | |
| fiber | | |
| Starch by amylases | | 9.00 |
| Cellulose by cellulase | | 14.00 |
| digestion | | |
| Hemicellulose by | | 22.00 |
| difference | | |

### Uses of the Palm Fiber-Based Dietary Composition

According to the present invention, the palm trunk, juice, dietary supplements, and other compositions derived from the palm tree can be used to treat, reverse, and/or protect against the deleterious effects of free radicals and oxidative stress.

### I. Free Radicals and Oxidative Stress

Over the past few decades, free radicals, highly reactive and thereby destructive molecules, have come to be appreciated increasingly for their importance to human health and disease. Many common and life-threatening human diseases, including atherosclerosis, cancer, and aging, have free radical reactions as an underlying mechanism of injury.

A free radical is a molecule with one or more unpaired electrons in its outer orbital. Many of these molecular species are oxygen (and sometimes nitrogen) centered. Indeed, the molecular oxygen we breathe is a free radical. These highly unstable molecules tend to react rapidly with adjacent molecules, donating, abstracting, or even sharing their outer orbital electron(s). This reaction not only changes the adjacent, target molecule, sometimes in profound ways, but often passes the unpaired electron along to the target, generating a second free radical or other ROS, which can then go on to react with a new target. In fact, much of the high reactivity of ROS is due to their generation of such molecular chain reactions, effectively amplifying their effects many fold. Antioxidants afford protection because they can scavenge ROS before they cause damage to the various biologbal molecules, or prevent oxidative damage from spreading, e.g., by interrupting the radical chain reaction of lipid peroxidation.

### ROS and Human Health

Because our bodies are continuously exposed to free radicals and other ROS, from both external sources (sunlight, other forms of radiation, pollution) and generated endogenously, ROS-mediated tissue injury is a final common pathway for a number of disease processes. The palm fiber compositions described herein protect a mammal against damage due to ROS, when the palm fiber compositions are administered to the mammal as described.

### Radiation Injury

Radiation injury represents an important cause of ROS-mediated disease. Extreme examples include the physical-chemical reactions within the center of the sun and at the center of a thermonuclear blast. With respect to more commonly encountered levels of radiation, depending upon the situation, about two-thirds of the sustained injury is mediated not by the radiation itself, but by the ROS generated secondarily. This applies not only to the acutely toxic forms of radiation injury, but the long-term, mutagenic (and hence carcinogenic) effects as well.

An important clinical application of this principle is encountered regularly in the treatment of cancer by radiation therapy. Large tumors often outgrow their blood supplies and tumor cells die within the center, despite being well-oxygenated at the periphery. Between these two regions is an area of tumor that is poorly oxygenated, yet remains viable. Radiation therapy of such tumors is particularly effective at the periphery, where an abundant concentration of oxygen is available to form tumorcidal ROS. The poorly oxygenated center is injured to a significantly smaller degree. While the dead cells in the center don't survive anyway, the poorly oxygenated, yet viable, cells between these two areas can survive a safe dose of radiation therapy, and thereby seed a later local recurrence of the tumor. This is a major reason why many large tumors are treated by a combination of radiation therapy (to kill the tumor at its advancing edges) and surgical removal of the bulk of the tumor, including these particularly dangerous remaining cells, The palm fiber compositions described herein protect a mammal against damage due to ROS, when the palm fiber compositions are administered to the mammal as described, and thereby provide benefits for cancer patients undergoing radiation therapy.

### Cancer and Other Malignancies

Cancer and other malignancies all entail unconstrained cell growth and proliferation based upon changes in the cell's genetic information. In most cases, for example, one or more genes that normally constrain cell growth and replication is/are mutated, or otherwise inactivated. These genetic deficiencies correspond directly with deletions and sequence changes in the genetic code, resident in the cell's DNA. A frequently seen final common cause of such DNA damage is free radical injury. Of the myriad injuries sustained by our DNA on a daily basis, most are repaired by normal DNA repair mechanisms within the cell, while some result in cell death. Since such injuries are sporadic and distributed somewhat randomly across the genome, most lethal DNA injuries are clinically inconsequential, resulting in the loss of a few cells among millions. However, when a single cell sustains an injury that impairs growth regulation, it can proliferate disproportionately and grow rapidly to dominate the cell population by positive natural selection. The result is a tumor, frequently a malignant one, where the constraint of growth and proliferation is particularly deficient. Therefore, free radical injury to the genetic material is a major final common pathway for carcinogenesis. The palm fiber compositions described herein protect a mammal against damage due to free radical injury, when the palm fiber compositions are administered to the mammal as described, and thereby provide for the prevention and treatment of cancer in mammals.

ROS can be generated within the cell not only by external sources of radiation, but also within the body as a byproduct of normal metabolic processes. An important source of endogenous free radicals is the metabolism of some drugs, pollutants, and other chemicals and toxins, collectively termed xenobiotics. While some of these are directly toxic, many others generate massive free radical fluxes via the very metabolic processes that the body uses to detoxify them. One example is the metabolism of the herbicide paraquat. At one time, drug enforcement authorities used this herbicide to kill marijuana plants. Growers realized they could harvest the sprayed crop before it wilted, and still sell the paraquat-laced product. Many who smoked this product subsequently died of a fulminant lung injury. Fortunately, this approach has been abandoned as a particularly inhumane way to solve the drug problem.

While the paraquat story is a particularly striking example of a metabolic mechanism of free radical toxicity, many commonly encountered xenobioics, including cigarette smoke, air pollutants, and even alcohol are toxic, and often carcinogenic to a large degree by virtue of the free radicals generated by their catabolism within our bodies. Moreover, there is accumulating evidence that a diet rich in fruits and vegetables, which are high in natural antioxidants, and low in saturated fat (a particularly vulnerable target for damage by ROS), reduces the risk of atherosclerosis and cancer.

### Atherosclerosis

Atherosclerosis remains the major cause of death and premature disability in developed societies. Moreover, current predictions estimate that by the year 2020 cardiovascular diseases, notably atherosclerosis, will become the leading global cause of total disease burden, defined as the years subtracted from healthy life by disability or premature death. Atherosclerosis is a complex process that leads to heart attack, stroke, and limb loss by the plugging of the arteries with atherosclerotic plaque. This plaque is a form of oxidized fat. When free radicals react with lipids, the consequence is lipid peroxidation, the same process by which butter turns rancid when exposed to the oxygen in the air. While a number of factors influence the development and severity of atherosclerosis, a major factor is the ROS-mediated peroxidation of our low density lipoproteins (LDLs, or “bad cholesterol”). The dietary approach to the prevention of heart disease and stroke is based partially on adding dietary antioxidants to limit LDL oxidation, as well as decreasing the intake of fat itself. These approaches already have made significant inroads into the mortality from heart disease, but the compositions of the present invention may offer a safe pharmacological prevention in the future that is not as dependent upon willpower as are diet and exercise. The palm fiber compositions described herein protect a mammal against damage due to lipid peroxidation, when the palm fiber compositions are administered to the mammal as described, and thereby provide for the prevention and treatment of atherosclerosis in mammals.

### Neurological and Neurodegenerative Diseases

Neurological and neurodegenerative diseases affect millions of Americans. These include depression, obsessive-compulsive disorder, Alzheimer's, allergies, anorexia, schizophrenia, as well as other neurological conditions resulting from improper modulation of neurotransmitter levels or improper modulation of immune system functions, as well as behavioral disorders such as ADD (Attention Deficit Disorder) and ADHD (Attention Deficit Hyperactivity Disorder). A number of these diseases appear to have ROS toxicity as a central component of their underlying mechanism of nerve cell destruction, including, but not limited to, amyotrophic lateral sclerosis (ALS, or Lou Gehrig's disease), Parkinson's disease, and Alzheimer's disease. The palm fiber compositions described herein protect a mammal against damage due to ROS toxicity, when the palm fiber compositions are administered to the mammal as described, and thereby provide for the prevention and treatment of the above mentioned diseases in mammals.

### Ischemia/Reperfusion Injury

When an organ is deprived of its blood supply (ischemia) it is injured, not just by the temporary loss of oxygen, but also by the ROS that are generated by reaction with the oxygen that is reintroduced at reperfusion, when the blood supply is restored. In some clinical situations, this injury can prevented by giving antioxidants, sometimes even after the period of ischemia, but just prior to reperfusion. For example, the preservation of kidneys, livers, and other organs in solutions that contain antioxidants, as well as other agents, is now routine prior to their transplantation. Another example is the use of drugs that block the function of free radical generating enzymes prior to stopping the heart for cardiac surgery. These drugs help prevent reperfusion injury when the heart is restarted and flow is restored. This reperfusion injury mechanism also has been found to play an important role in patients suffering from multiple organ failure after trauma, massive surgery, or shock. Multiple organ failure is now the leading cause of death in intensive care units, and extensive efforts are under way to understand better how ROS contribute to this syndrome. The palm fiber compositions described herein protect a mammal against damage due to ROS toxicity, when the palm fiber compositions are administered to the mammal as described, and thereby provide for the prevention and treatment of the above mentioned diseases in mammals.

### Aging

Aging is a remarkably complex process that has managed to remain relatively opaque to scientific understanding. There is now evidence that aging is a series of processes, i.e., a series of controlled mechanisms, and not just the passive accumulation of wear and tear over the years. If aging is a series of processes, some of these processes are potentially controllable, or at least modifiable. One of the most important of these processes is comprised of an accumulation of the molecular injuries that are mediated by free radicals and other ROS. Recent studies indicate that the therapeutic manipulation of ROS metabolism can actually extend the total life span of mice to a significant degree. The palm fiber compositions described herein protect a mammal against damage due to ROS toxicity, when the palm fiber compositions are administered to the mammal as described, and thereby provide for anti-aging effects in mammals.

### II. Other Uses of the Palm Fiber-Based Dietary Composition of the Invention

As detailed below, palm fiber-based dietary compositions of the invention are useful in the prevention and treatment of a variety of disorders, e.g., but not limited to, inflammatory bowel disease, high cholesterol, gastrointestinal disorders; diabetes and cancer (See generally, Innami and Shimizu, Dietary Fiber and Fecal Characteristics in Humans and Animals, In Food Factors for Cancer Prevention, Eds. Ohigashi, et al., Springer; Eastwood M (1990) Fiber and gastrointestinal disease. In: Kritchevsky D. Bonfield C. Anderson J W (eds) Dietary fiber, chemistry, physiology and health effect. Plenum, New York, pp 261-271; Ohta et al., (in Japanese). Jpn J Gastroenterology 82:51-57 (1985); Harashima E, Tsuji K, Nakagawa Y, Urata G (1994); Saito et al., J Nutr Sci Vitaminol 37:493-508 (1991); Ohta M, (in Japanese). J Jpn Soc Coloproctol 40:741-746 (1987); Cummings et al., Gastroenterology 103:1782-1789 (1992): Hill W J (1986). Bile acids and colorectal cancer in humans. In: Vahouny G V, Kritchevsky D (eds) Dietary fiber—basic and clinical aspects. Plenum, New York, pp 497-513; Narisawa et al., J Natl Cancer Inst. 64:573-578 (1974); Wilpart et al, Carcinogenesis 4:45-48 (1983); Sannoumaru et al., J Nutr Sci Vitaminol J Nutr Sci Vitaminol (Tokyo), April; 42(2):97-110, (1996); Cummings J H (1978) Diet and transit through the gut. In: Heaton K W (ed) Dietary fiber—current development of importance to health. John Libbey, London, pp 83 95; J Home Econ Jpn 45:1079-1087. Low fiber intake can lead to a deficiency of colonic functions and many physiological problems.

Mature palm trunk, e.g., 20 year-old oil palm tree, is high in lignin. Lignin is a component of fiber that undergoes minimal changes in the body and is able to bind cholesterol, bile salts, fats, carbohydrates and toxins. The palm fiber-based dietary compositions of the invention are useful to, e.g., relieve constipation, maintain a healthy digestive system, normalizing the balance of beneficial and pathological bacteria in the colon, normalizing blood sugar level, decrease inflammation of the bowel, lower blood cholesterol level, lower colon cancer risk and lower breast cancer risk; when the palm fiber compositions are administered to the mammal as described, and thereby provide for the prevention and treatment of the above mentioned diseases in mammals.

### Cancer

Dietary fibers have proven themselves a key element in preventing and impairing the progression of cancerous cells. Two major cancers, breast cancer and colon cancer can be effectively prevented by simple increasing the intake of dietary fiber. The palm fiber composition of the present invention can demonstrate similar prophylactic and impairment effects on the progression of cancerous cells when the palm fiber compositions are administered to the mammal as described, and thereby provide for anti-cancer effects in mammals.

### Colon Cancer

Dietary fiber is a very effective agent in preventing the development of colon cancer. Pathological bacteria in the colon can be a cause of colon cancer, due to their production of toxins that can damage the cells and it DNAs. Fiber normalizes bowel transit time, relieves constipation, and binds to toxins. These will minimize the toxins that are in contact with the intestinal wall, thus minimizing the chance of colon cancer.

Bile salts are also known to be carcinogenic, the binding of bile salts to dietary fiber, minimizes the contact of this carcinogen o the colon wall, reducing the chance of developing colon cancer.

Beneficial bacterial in the colon is able to digest lignin which exits in Oil Palm Trunk Dietary Fiber. These bacteria will produce what is called the mammalian lignan, which increases resistance to infection and development of cancer. Some bacteria also produce one particular fatty acid, butyrate, this prevents certain genes from being switched on and cause colon cancer.

The palm fiber composition of the present invention can demonstrate similar prophylactic effects for colon cancer.

### Breast Cancer

Dietary fiber can reduce the production of oestrogen in the body. High level of this hormone is responsible for causing and the progression of breast cancer. Reduction of oestrogen level in the patients and early states of breast cancer has shown to facilitate its regression. Fiber also provides nutrition to your bowel flora—the friendly microorganisms in your intestines that work to promote your health. If these bacteria are fed well with fibers that reproduce in greater numbers and produce a substance called mammalian lignan, which increases resistance to infectious agents and the cancer.

Lignans are converted into weak oestrogen in the intestines, and they complete with body oestrogen, which in more potent and more carcinogenic, for binding sites in the breast. High fiber diet is also associated with low fat diet and slimmer body. These are associated with lower level of oestrogen in the female body. These effects help reduce the risk of developing breast cancer.

Sufficient Intake of fiber which is rich in lignin is proven to reduce the risk of infectious agents and cancer. Breast cancer patients, and others at high risk of colon and breast cancer, excrete far fewer lignans (produced by the beneficial bacteria in our intestine from lignin) than healthy people. This implies that they have fewer lignans present in their bowel than do people without these types of cancers.

The palm fiber composition of the present invention can demonstrate similar prophylactic effects for breast cancer.

### Constipation

Perhaps the most frequently reported bowel problem that people experience is constipation. The best way to tackle the problem is with dietary fiber, and it is the natural way to solving this problem without resolving to medication and laxatives.

Insoluble fiber, softens fecal matter by retaining moisture and increases the bulk and consistency of the fecal matter. Thus stimulates the intestine and facilitate the movement of the fecal matter. Poorly formed feces lengthen the time feces stay in the rectum as it does not stimulate the rectum enough, this can have significant consequences that include, e.g., water content in the fecal matter is extracted and the feces become very hard and causes constipation. These hard feces might even damage the intestinal wall; bacteria in the colon will feast on these over stayed feces and produces toxins; toxins can be absorbed by the body, and cause health issues. Retained feces even after opening the bowel. As the feces are poorly formed, some can be left behind in the rectum, giving an uncomfortable sensation of incomplete voiding. These retained product will be feasted on by bacteria and produce toxins. During evacuation, hard stools place a strain on the colonic muscles and on the lining of the lower portion of the large intestine, rectum and anus. Straining causes many problems that include, e.g., hemorrhoids, diverticula disease of the colon (formation of pouch in the wall of the intestinal wall, which can be infected and even rupture, causing peritonitis that is potentially lethal); varicose veins; and hernias. Finally, the much more serious and life-threatening problems of cancer and immune system disorders may begin inside a toxic bowel.

The palm fiber composition of the present invention can demonstrate similar prophylactic effects for constipation as well as irritation of bowel tissue.

### Bowel Transit Time

Dietary fiber also plays an important role in normalizing bowel transit time. The longer the toxic waste matter sits in the bowel, allowing proteins to putrefy, fats to become rancid, and carbohydrates to ferment. The longer your body is exposed to rotting food in your intestines, the greater your risk of developing diseases.

Because a lack of fiber causes slower movement of feces through your bowel, it allows carcinogenic substances to be in contact with your intestinal wall longer. This could lead to possible formation of colon cancer. Frequently, a blocked or slow-moving bowel can cause problems, e.g., lower back pain, neck and shoulder pain, wrist and hand pain, skin problems, ‘Brain fog”, fatigue, sluggishness, common headache, and neurological problems.

The palm fiber composition of the present invention can demonstrate similar effects for normalizing bowel transit time.

### Internal Cleansing

Another way in which dietary fibers maintain a health digestive system is its ability to cleanse the digestive system. Dietary fibers do not only reduce toxins produced by relieving constipation and normalizing bowel transit time, but it can also retain toxins produced by bacteria especially in the colon.

The digestive system has a very thin inner wall, but it has one of the highest cell turnover times. Every 4-5 days, the intestine will completely shed its inner wall cells. Dietary fiber aids the shedding of these cells, which are damaged, dead, beyond their functioning life, and are removed quickly along with their toxins, bacteria and chemical wastes from our body system.

The palm fiber composition of the present invention can demonstrate similar bowel cleansing effect.

### Cholesterol Levels

Studies have shown that fiber has a cholesterol-lowering effect on the blood. They lower the level of harmful LDL cholesterol in the body, while raising the valuable and protective HDL level. Fiber sweeps out toxic materials as it moves down the intestinal tract. The body uses the fiber sweep as a key way of ridding itself of cholesterol. The liver converts the cholesterol to bile salts and these are excreted into the intestinal tract. The fiber then helps sweep the bile salts out of the body. In short, dietary fiber is an important tool in elimination excess cholesterol. The palm fiber compositions described herein protect a mammal against vascular damage due to LDL and lipid peroxidation, when the palm fiber compositions are administered to the mammal as described, and thereby provide for anti-athlosclerotic effects in mammals.

### Blood Sugar Levels

Fiber slows the release of sugar into your bloodstream, which prevents and exhausting demand on the release of insulin. If you have normal pancreatic function, your body produces insulin in response to the sugar load in your bloodstream from food you have eaten. Insulin brings your blood levels back into a normal range.

Diabetics who cannot produce insulin from their pancreas must use medication in tablet form or by injection to normalize their blood sugar. As a benefit of adequate fiber intake, insulin-dependent diabetics may be able to reduce their required dose of insulin.

The palm fiber compositions described herein protect a mammal against damage due to altered blood sugar levels, when the palm fiber compositions are administered to the mammal as described, and thereby provide for anti-diabetic effects in mammals.

### Beneficial Bacteria and Pathological Bacteria

Bacteria in the intestine can be divided into two groups, the beneficial bacteria, that help the body digest food and fight infections, and pathological bacteria, that can cause diseases.

Beneficial bacteria, take Laciobacillus bifidus for example, is known to enhance food digestion and absorption, suppress the growth of pathological bacteria and the composition of some vitamins. It is therefore beneficial for us to preserve these bacteria. There is a wide range of pathological bacteria; the common types are bacteria like Campylobacter jejuni, E. coli, Salmonella, Clostridium perfringens, and so on and so forth. These bacteria not only compete with the host of food, but also produce a wide variety of toxins that can cause illnesses.

It is essential to promote the growth of beneficial bacteria and in the same time suppress the growth of pathological bacteria. Any disruption of the balance between the beneficial and pathological bacteria will lead to illnesses, ranging from small ailments to serious medical conditions or even death.

In order to maintain the balance of beneficial and pathological bacteria in the digestive system, we have to be careful of what we eat as this can have a great impact on these bacteria. Certain food promotes the growth of beneficial bacterial directly or indirectly, and certain foods will do the same for pathological bacteria. Of course, the consumption of unhygenic food will introduce other strains of bacteria into the digestive system, which could be pathological.

### Pharmaceutical Compositions and Formulations

The palm fiber-based dietary supplements of the present invention can be used in beverages, tonics, infusions, or food-stuffs alone, or in combination with other dietary supplements or therapeutics. The palm fiber-based dietary supplements of the invention can be used alone or further formulated with pharmaceutically acceptable compounds, vehicles, or adjuvants with a favorable delivery profile, i.e., suitable for delivery to a subject. Such compositions typically comprise the palm fiber-based dietary supplement of the invention and a pharmaceutically acceptable carrier. As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include oral, intravenous, intraperitoneal, subcutaneous, intramuscular, intraarticular, intraarterial, intracerebral, intracerebellar, intrabronchial, intrathecal, topical, and aerosol route. The pH can be adjusted with acids or bases, such as hydrochloric acid or sodium hydroxide.

Oral compositions generally include an inert diluent or an edible carrier. They can be enclosed in gelatin capsules, caplets or compressed into tablets. For the purpose of oral therapeutic administration, the palm fiber-based dietary supplements of the invention can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

The palm fiber-based dietary supplements of the invention can also be prepared as pharmaceutical compositions in the form of suppositories (e.g., with conventional suppository bases such as cocoa butter and other glycerides) or retention enemas for rectal delivery.

In one embodiment, the palm fiber-based dietary supplements of the invention are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the palm fiber-based dietary supplement and the particular therapeutic effect to be achieved, and the limitations inherent in the art of compounding such an active compound for the treatment of individuals. The pharmaceutical compositions can be included in a container, pack, or dispenser together with instructions for administration.

The invention is further defined by reference to the following examples, which are not meant to limit the scope of the present invention. It will be apparent to those skilled in the art that many modifications, both to the materials and methods, may be practiced without departing from the purpose and interest of the invention.

### EXAMPLES

### Example 1 Fecal Bulking Analysis of a Palm Fiber-Based Dietary Composition of the Invention

### I. General

A palm fiber composition of the invention, designated lot # MAL001, was incorporated into complete rat diets, and the relative fecal bulking potential determined as the fecal bulking index (FBI). The FBI is a measure of the increase in hydrated fecal mass over baseline due to an ingested material, as a percentage of the increase due to an equal weight of reference material.

### II. Procedure

Eight adult male Sprague-Dawley rats of 350-400 g weight were used per diet group, after preconditioning on diets containing mixed dietary fiber. The five diets that were analyzed included, AACC (American Association of Cereal Chemists) wheat bran reference; Fiberx sugar beet fiber (Fiber® sugar beet fiber, grade 595, <125 μm particle size, Danisco Sugar AB, MaIma, Sweden); Palm fiber-based dietary composition of the invention (lot # MAL001); In-house wheat bran reference (IH WB ref.) from locally grown (New Zealand) wheat; and Baseline (sucrose). All diets were based on the nutritionally complete baseline diet, which contained 50% sucrose. Test diets were made up by substituting test material for sucrose in the baseline πt formulation at an inclusion rate of 10%. The composition of the diets is summarized below in Table 9.

TABLE 9
Composition (g/kg) of diets used for determination
of the relative fecal bulking

| | AACC | palm fiber- | | | |
|---|---|---|---|---|---|
| | wheat | based dietary | | IH WB | |
| | bran ref. | composition | Fiberx ® | ref..* | Baseline |
| Complete | 500 | 500 | 500 | 500 | 500 |
| diet base | | | | | |
| Sucrose | 400 | 400 | 400 | 400 | 500 |
| Fiber source | 100 | 100 | 100 | 100 | — |

*IH WB ref.. = In-house wheat bran reference.

The rats were fed the diets for a seven-day period which included a three day clean-out and a four-day balance period during which the diets, spillage and refusal were measured, and fecal collections made. The feces were air-dried, freeze-dried and weighed, and a subsample of them rehydrated to obtain a rehydrated fecal weight, from which water retention capacity, hydrated fecal weight per 100 g diet, and the FBI were calculated. The FBI is calculated by dividing the increase in fecal bulk over baseline due to test material by the increase in fecal bulk over baseline due to reference and then multiplying the resultant value by a factor of 100. In addition to an FBI value, the FBI analysis provides information on fecal dry matter output, fecal water retention capacity, and fecal water load per 100 g diet, and the increases in these parameters over baseline.

### III. Results

Table 10 shows that Palm Fiber lead to a greater fecal dry matter output than the other dietary fiber sources tested.

TABLE 10
Fecal Dry matter/100 g diet

| | | | | palm | |
|---|---|---|---|---|---|
| | | AACC | | fiber-based | |
| | | wheat | | dietary | |
| | Baseline | bran | Fiberex | composition | IH WB ref. |
| Mean ± SD | 6.61 ± | 11.61 ± | 9.54 ± | 14.09 ± 0.49 | 12.11 ± 0.57 |
| | 0.24 | 0.63 | 0.46 | | |

Table 11 shows that the water retention capacity per gram of fecal dry matter was greater in the Palm Fiber group than in the comparison groups.

TABLE 11
Water retention capacity g/g dry matter

| | | | | palm | |
|---|---|---|---|---|---|
| | | | | fiber-based | |
| | | AACC | | dietary | |
| | Baseline | Bran | Fiberex | composition | IH WB ref. |
| Mean ± SD | 2.31 ± | 2.88 ± | 2.85 ± | 4.07 ± 0.18 | 2.97 ± 0.18 |
| | 0.11 | 0.17 | 0.30 | | |

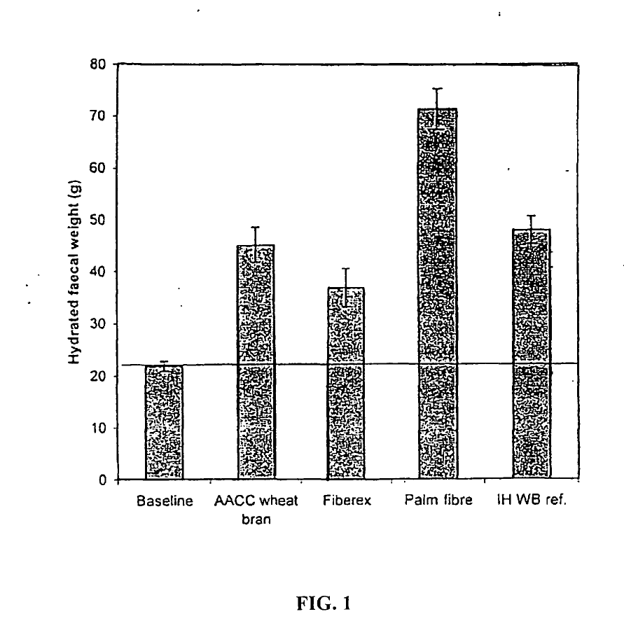

As shown in Table 12 and FIG. 1, when the dry matter output and water retention capacity of the fecal outputs were combined and expressed per 100 g diet the palm fiber-based dietary composition of the invention had a much greater capacity to augment hydrated fecal bulk than the other materials tested.

TABLE 12
Hydrated fecal output/100 g diet

| | | | | palm | |
|---|---|---|---|---|---|
| | | AACC | | fiber-based | |
| | | wheat | | dietary | Wheat |
| | Baseline | bran | Fiberex | composition | bran ref. |
| Mean ± SD | 21.9 ± 0.9 | 45.0 ± 3.4 | 36.8 ± | 71.4 ± 4.0± | 48.1 ± 2.7 |
| | | | 3.7 | | |

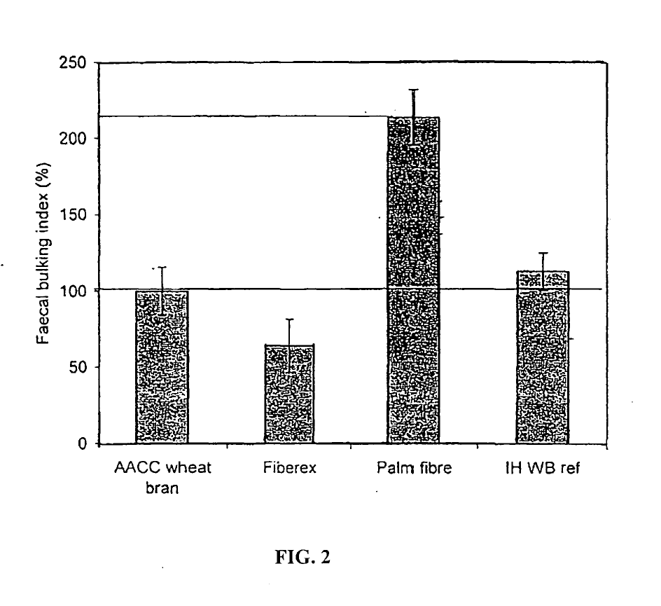

The FBI values summarized in Table 13 were obtained by expressing the data presented in Table 7 as the increase over baseline due to the test material as a percent of the increase due to the AACC wheat bran reference. As shown in Table 13 and FIG. 2, the palm fiber-based dietary composition of the invention was about twice as effective as wheat bran as a fecal bulking material.

TABLE 13
Fecal bulking index (%) using AACC wheat bran
(FBI = 100) as reference

| | | | palm | |
|---|---|---|---|---|
| | AACC | | fiber-based dietary | |
| | wheat bran | Fiberex | composition | IH WB ref. |
| Mean ± SD | 100 ± 16 | 64 ± 17 | 214 ± 18 | 113 ± 12 |

The theoretical fecal water load per 100 g diet was calculated based on the fecal dry matter per 100 g diet and the water retention capacity of the fecal matter. The values for the theoretical fecal water load per 100 g diet are summarized below in Table 14.

TABLE 14
Colonic water load/100 g diet

| | | | | palm | |
|---|---|---|---|---|---|
| | | AACC | | fiber-based | |
| | | wheat | | dietary | IH WB |
| | Baseline | bran | Fiberex | composition | ref. |
| Mean ± | 15.3 ± 0.8 | 33.4 ± 2.9 | 27.2 ± 3.4 | 57.3 ± 3.6 | 36.0 ± 2.4 |
| SD | | | | | |

The results in Table 14 expressed as the % increase in water load over baseline are shown in Table 15. Table 15 shows that 10% Palm Fiber might lead to an almost three fold increase in water flux through the colon. That is, while 10% wheat bran doubled water load, the Palm Fiber quadrupled it.

TABLE 15
Percent increase in fecal water load/100 g
diet due to 10% fiber inclusion

| | AACC | | Palm fiber-based | IH WB |
|---|---|---|---|---|
| | wheat bran | Fiberex | dietary composition | ref. |
| Mean ± | 119.0 ± 19.2 | 78.2 ± 22.5 | 275.3 ± 23.5 | 135.5 ± 15.4 |
| SD | | | | |

Based on the fecal bulking effects and water retention capacity of the sample provided, the palm fiber-based dietary composition of the invention are useful as a food ingredient for health foods. The palm fiber-based dietary composition of the invention; survives colonic fermentation and retain its capacity to retain water to increase fecal bulk.

### EQUIVALENTS

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

1.-16. (canceled)

17. A therapeutic method comprising:
administering to a mammal, a composition having an effective amount of purified tissue of African Oil Palm; wherein the composition provides an antioxidant effect and beneficial fiber to the mammal.

18. The method of claim 17, wherein administering the composition also acts as an inhibitor of active oxygen production.

19. The method of claim 17, wherein administering the composition also protects against free radicals, reactive oxygen scavenger (ROS), ROS-mediated peroxidation, and oxidative damages.

20. The method of claim 17, wherein administering the composition also protects against damages due to hydroxyl radiation, nitric oxide, superoxide, and peroxynitrite.

21. The method of claim 17, wherein administering the composition also provides prophylactic effects for constipation and irritation of bowel tissue, normalizing bowel transit time, and cleansing of the digestive system.

22. The method of claim 17, wherein administering the composition also protects against vascular damage due to LDL and lipid peroxidation, diabetic effects due to altered blood sugar, imbalances beneficial and pathological bacteria within the digestive system.

23. A method of making an antioxidant preparation, the method comprising:
a) obtaining purified tissue of African Oil Palm; and
b) processing the purified tissue, such that it is suitable for administration to a mammal; wherein the purified tissue provides an antioxidant effect and beneficial fiber to the mammal.

24. The method of claim 23, wherein the African oil palm is obtained from trunk tissue.

25. The method of claim 23, wherein the purified tissue is at least 10% by weight of the preparation.

26. The method of claim 23, wherein the purified tissue is 50% to 80% by weight of the preparation.

27. The method of claim 23, wherein the purified tissue is 60% by weight of the preparation.

28-43. (canceled)

44. A method of treating a subject, the method comprising:
administering to the subject an effective amount of a composition comprising purified tissue of African Oil Palm suitable for administration to a mammal and a pharmaceutically acceptable carrier, wherein the purified tissue of African Oil Palm is provided with the pharmaceutically acceptable carrier.

45. The method of claim 44, wherein the composition inhibits active oxygen production.

46. The method of claim 44, wherein the composition protects against free radicals, reactive oxygen scavenger (ROS), ROS-mediated peroxidation, and oxidative damages.

47. The method of claim 44, wherein the composition protects against damages due to hydroxyl radiation, nitric oxide, superoxide, and peroxynitrite.

48. The method of claim 44, wherein the composition provides prophylactic effects for constipation and irritation of bowel tissue, normalizing bowel transit time, and cleansing of the digestive system.

49. The method of claim 44, wherein the composition protects against vascular damage due to LDL and lipid peroxidation, diabetic effects due to altered blood sugar, imbalances beneficial and pathological bacteria within the digestive system.
